(19) United States
(12) Patent Application Publication
(10) Pub. No.: US 2026/0215685 A1
(43) Pub. Date: Jul. 30, 2026

(54) POST-OPERATIVE TORIC IOL EVALUATION SYSTEM

(71) Applicant: Alcon Inc., Fribourg (CH)

(72) Inventors: John Alfred Campin, Southlake, TX (US); George Hunter Pettit, Fort Worth, TX (US); Joseph Weatherbee, San Clemente, CA (US)

(21) Appl. No.: 19/404,946

(22) Filed: Dec. 1, 2025

Related U.S. Application Data
(60) Provisional application No. 63/751,310, filed on Jan. 30, 2025.

(51) Int. Cl. A61B 3/18; A61B 3/00; A61B 3/10; A61B 3/103; A61B 3/107; G06T 7/00

(52) U.S. Cl. CPC A61B 3/18; A61B 3/0008; A61B 3/0025; A61B 3/1015; A61B 3/102; A61B 3/1035; A61B 3/107; G06T 7/0012; G06T 2207/10004; G06T 2207/10024; G06T 2207/10101; G06T 2207/20; G06T 2207/30041

(57) ABSTRACT

One or more images of an eye of a patient having an implanted IOL are received. The one or more images are captured using one or more imaging devices having one or more imaging modalities. A computing device determines misalignment of the IOL according to the one or more images and processes the misalignment using a predictive model to obtain an improvement probability. The computing device outputs an intervention recommendation according to the improvement probability. The intervention recommendation may be the result of cost/benefit analysis of the improvement probability.

### TECHNICAL FIELD

The present disclosure relates generally to methods for the treatment of cataracts and, more particularly, to treatments including the use of intraocular lenses (IOL).

### BACKGROUND

Light received by the human eye passes through the transparent cornea covering the iris and pupil of the eye. The light is transmitted through the pupil and is focused by a crystalline lens positioned behind the pupil in a structure called the capsular bag. The light is focused by the lens onto the retina, which includes rods and cones capable of generating nerve impulses in response to the light.

Through age or disease, the crystalline lens may become cloudy, a condition known as a cataract. Cataracts are a readily treated by removing the crystalline lens and inserting an artificial lens, known as an intraocular lens (IOL). The IOL may be fabricated to additionally correct for aberrations of the patient’s eye, such as astigmatism. Inasmuch as astigmatism is the result of asymmetry of the eye, the IOL must be aligned with the asymmetry of the eye in order to compensate for it. The IOL is therefore provided with markers, such as rows of dots at the perimeter of the IOL, which define an axis that may be used to align the IOL. The IOL may be implemented as a toric IOL, which includes spring-like arms, known as haptics, which hold the IOL in place within the capsular bag. In prior approaches, an imaging device, such as a digital marker microscope (DMM), is used to view the patient’s eye during surgery. The image output by the imaging device has a reference axis superimposed thereon that corresponds to the desired orientation of the axis of the IOL.

Inasmuch as precise alignment of the IOL axis with the reference is desired, approaches for facilitating this alignment would greatly improve patient outcomes.

### BRIEF SUMMARY

The present disclosure relates generally to a system for evaluating an intraocular lens (IOL), such as a toric IOL, in a patient’s eye.

Particular embodiments disclosed herein provide a method including receiving, by a computing device, one or more images of an eye of a patient having an implanted IOL, the one or more images captured using one or more imaging devices having one or more imaging modalities. The computing device determines misalignment of the IOL according to the one or more images and processes the misalignment using a predictive model to obtain an improvement probability. The computing device outputs an intervention recommendation according to the improvement probability.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates anatomy of a human eye.

FIG. 2 illustrates an example toric intraocular lens (IOL).

FIG. 3 illustrates a process of aligning a toric IOL with respect to a reference axis, in accordance with certain embodiments.

FIG. 4 illustrates post-operative imaging of an eye having a toric IOL placed therein, in accordance with certain embodiments.

FIG. 5 illustrates a method for performing post-operative evaluation of placement of a toric IOL, in accordance with certain embodiments.

FIG. 6 illustrates a system for evaluating suitability of post-operative intervention, in accordance with certain embodiments.

FIG. 7 illustrates an example computing device that implements, at least partly, one or more functionalities for performing post-operative evaluation of placement of an IOL, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Particular embodiments of the present disclosure provide an alignment guide for positioning a toric intraocular lens (IOL) in a patient’s eye.

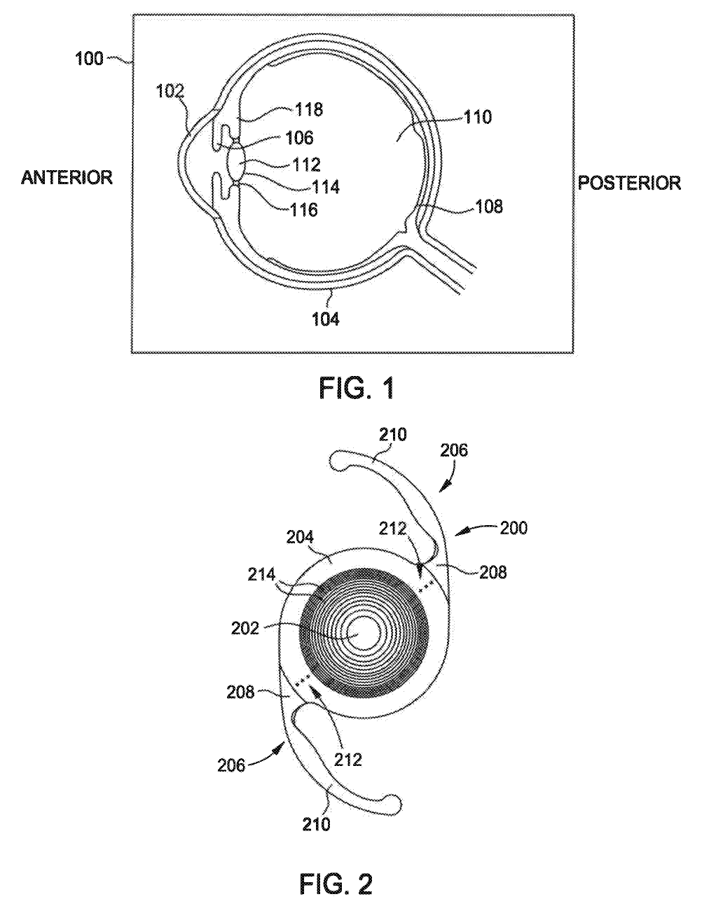

FIG. 1 is a diagram illustrating parts of the human eye 100 that may be understood with respect to the anterior side, through which light enters the eye, and the posterior side opposite the anterior side. At the anterior side of the eye 100, a thin transparent layer known as the cornea 102 is linked to the sclera 104, which forms the generally spherical wall of the eye 100. The cornea 102 and sclera 104 are connected by a ring called the limbus. The iris 106, the color of the eye, and an opening defined by it, the pupil, are positioned behind the cornea and are visible due to the cornea’s 102 transparency. The retina 108 is formed on an interior surface of the sclera 104 opposite the cornea 102 and iris 16. The volume defined by the sclera 104 is occupied by the transparent jelly of the vitreous body 110.

The crystalline lens 112 is a transparent, biconvex structure in the eye that, along with the cornea 102, helps to refract light to be focused on the retina 108. The lens 112, by changing its shape, functions to change the focal distance of the eye so that it can focus on objects at various distances, thus allowing a sharp real image of the object of interest to be formed on the retina 108. This adjustment of the lens 112 is known as accommodation, and is similar to the focusing of a photographic camera via movement of its lenses.

The lens 112 is positioned behind the iris 106 in a capsular bag 114. The capsular bag 114 is attached at its perimeter to the suspensory ciliary ligament 116. The ciliary ligament 116 attaches the capsular bag 114 to the ciliary body 118. The ciliary body 118 is a ring-shaped muscle that attaches the ciliary ligament 116 to the sclera 104 and which can contract or relax in order to change the shape of the lens 112.

Various diseases and disorders of the lens 112 may be treated with an IOL. By way of example, not necessarily limitation, an IOL according to embodiments of the present disclosure may be used to treat cataracts, large optical errors in myopic (near-sighted), hyperopic (far-sighted), and astigmatic eyes, ectopia lentis, aphakia, pseudophakia, and nuclear sclerosis. However, for purposes of description, the IOL embodiments of the present disclosure are described with reference to cataracts, which often occurs in the elderly population.

FIG. 2 illustrates an example toric IOL 200. The toric IOL 200 includes a lens portion 202 that focuses light passing through the iris 106 onto the retina 108. The lens portion 202 may be surrounded by a peripheral ring 204 that is not used to focus light. Two or more haptics 206 may secure to the peripheral ring 204. Each haptic 206 may include a base 208 secured to the peripheral ring 204 and extending outwardly therefrom. A spring arm 210 secures to the base and extends both outwardly from the peripheral ring 204 and circumferentially around the peripheral ring 204. In use, the spring arms 210 push outwardly against the capsular bag 114 and hold the toric IOL 200 in a desired position.

Marks 212 may be formed on the peripheral ring 204. The marks 212 facilitate alignment of the IOL with the eye 100 of the patient. The marks 212 in the illustrated toric IOL 200 include two sets of dots (e.g., depressions or bumps), such as circular dots, formed on the peripheral ring 204 opposite one another. For example, each set may include, two, three, or more dots. The dots of each set may be collinear with one another and be collinear with the dots of the other set. Some toric IOLs 200 are multi-focal. The lens portion 202 may include rings 214 that define the boundary between regions of the lens portion 202 with different focal lengths.

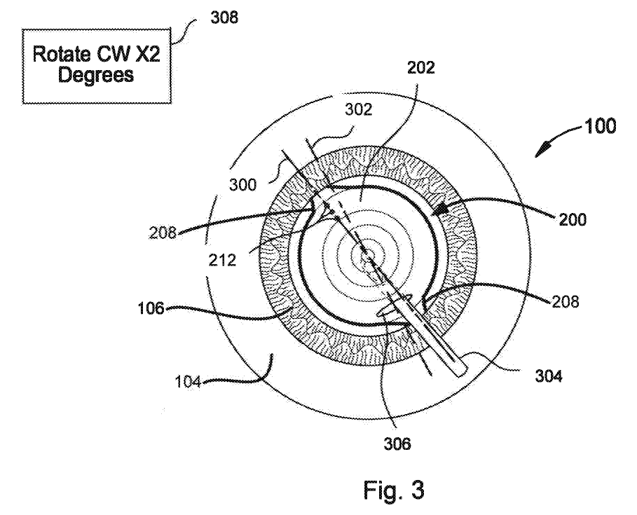

Referring to FIG. 3, a line passing through the marks 212 of one or both sets (hereinafter “the IOL axis” 300) may also intersect and be perpendicular to the optical axis of the lens portion 202. In the illustrated toric IOL 200, the IOL axis 300 also intersects the bases 208 of the haptics 206. The marks 212 may be detected in images using a machine learning model and used to determine the IOL axis 300. Accordingly, the marks 212 need not be intersected by the IOL axis 300 and may include any arbitrary pattern that is visible in an image of the IOL 200. The machine learning model may be trained to identify the marks 212 of whatever shape, arrangement, and number. Features other than the marks 212 may be used to determine the orientation of the IOL 200, such as the bases 208 of the haptics, the perimeter of the IOL 200. The geometrical relationships between any two or more features may be used to determine the orientation of the IOL 200 using a machine learning model.

The orientation of the eye 100 may be determined using a same or different machine learning model. Features of the sclera 104, iris 106, and/or retina 108 visible in an image may be compared with a reference image labeled with a reference axis 302. The image may be registered with respect to the reference image to determine the orientation of the eye 100 in the image and determine the orientation of a reference axis 302 of the eye 100 in the image. The reference axis 302 defines an orientation of the IOL axis 300 for which the IOL 200 will provide the most astigmatism correction. The smaller the angular difference between the IOL axis 300 and the reference axis 302, the lower the refractive error of the eye 100 following implantation of the IOL 200.

During implantation, a surgeon may insert an instrument 304 through an opening 306, such as an opening in the limbus of the eye 100, and engage the IOL 200 with the instrument 304 in order to rotate the IOL 200 and align the IOL axis 300 with the reference axis 302. Images from a surgical microscope that are labeled with the reference axis 302 may be displayed to the surgeon during this procedure in order to facilitate alignment. The IOL axis 300 may also be labeled in the images displayed to the surgeon. A graphical indicator 308 may be displayed on the images, which indicates the angular error between the reference axis 302 and the IOL axis 300.

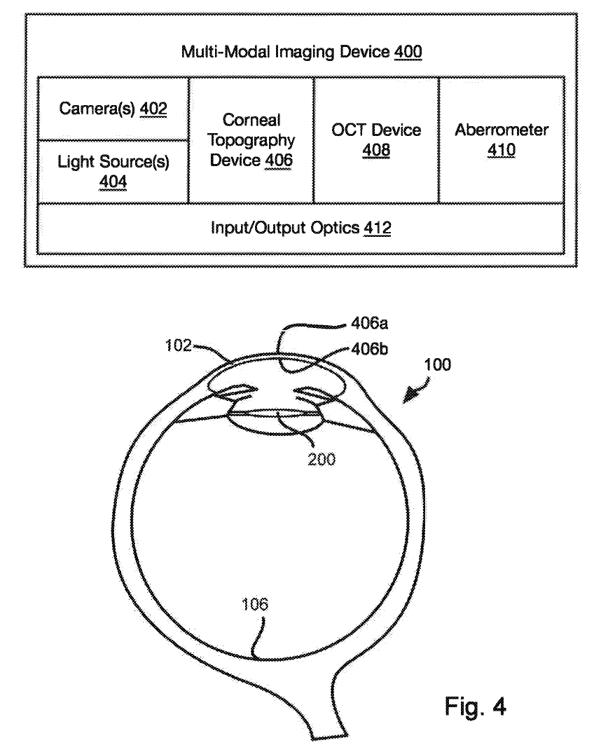

Referring to FIG. 4, post operative imaging of the eye 100 with the implanted IOL 200 may be performed using the illustrated multi-modal imaging device 400 or two or more separate imaging devices collectively performing the functions ascribed herein to the multi-modal imaging device 400.

The multi-modal imaging device 400 may include one or more cameras 402, such as a visible light camera. One or more light sources 404 may illuminate the eye 100 to facilitate capturing images with the one or more cameras 402. The one or more cameras 402 may be two cameras providing binocular vision. For example, the one or more cameras 402 and one or more light sources 404 may be implemented as the NGENUITY 3D VISUALIZATION SYSTEM provided by Alcon Inc. of Fort Worth Texas.

The multi-modal imaging device 400 may include a corneal topography device 406. The corneal topography device 406 measures the shape of the cornea 102 in order to estimate the diffractive power of the cornea 102 and any refractive error of the cornea 102, e.g., astigmatism. The corneal topography device 406 may measure the contours of the inner and outer surfaces 406a, 406b of the cornea 102 in order to perform the function thereof.

The multi-modal imaging device 400 may include an optical coherence tomography (OCT) device 408. The OCT device 408 obtains a volumetric image of the eye 100, including of one or both of the anterior chamber (region between the cornea 102 and the capsular bag 114) and the retina 108.

The multi-modal imaging device 400 may include an aberrometer 410, such as a wavefront aberrometer, that is configured to measure refractive error of the eye 100, including the combined refractive properties of the cornea 102, IOL 200, and the axial length of the eye 100.

The various imaging devices of the multi-modal imaging device 400 may use input/output optics 412 to transmit light to the eye 100 and receive light reflected from the eye. The input/output optics 412 may include one or more lenses and/or beam splitters for routing light to the various imaging devices. Alternatively, each imaging device may have its own input/output optics.

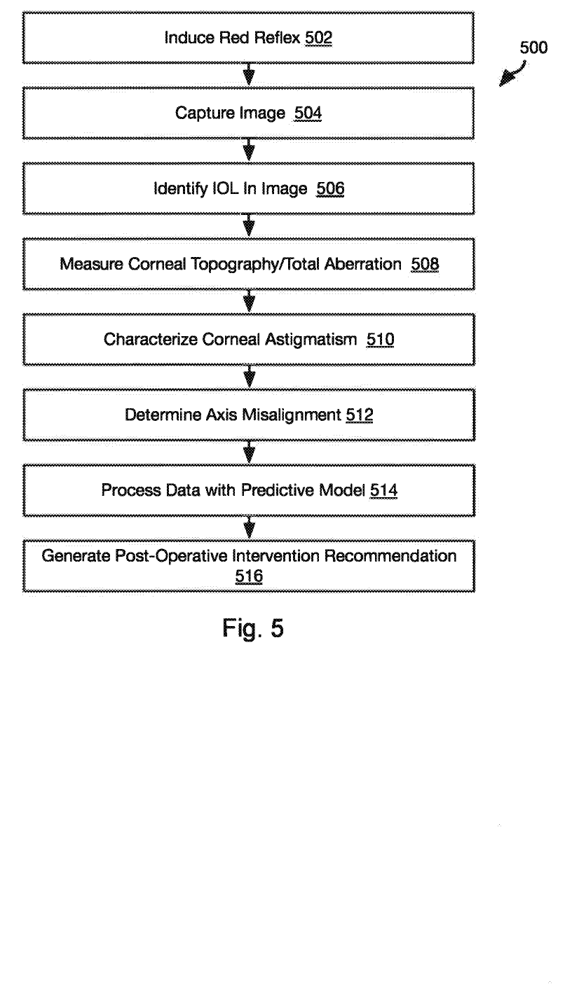

FIG. 5 illustrates a method 500 that may be used to perform post-operative evaluation of placement of an IOL 200. The method 500 may be performed using a computing system 700 and/or a computing device incorporated into the multi-modal imaging device 400.

The method 500 may include inducing, at step 502, red reflex of the eye 100. The red reflex is a reflection of primarily red light from the retina 108 that provides back lighting of the IOL 200 and facilitates visualization of the marks 212 and other features of the IOL 200. Inducing red reflex may include emitting light from the one or more light sources 404 to promote the red reflex, such as light that is primarily red, e.g., having a peak intensity at 700 nm +/- 50 nm.

The method 500 may include capturing an image of the eye 100 at step 504. The IOL 200 may be identified in the image, including identifying the orientation thereof. Identifying the IOL 200 in the image may be performed using one or more machine learning models trained to perform this task. For example, the machine learning model may be trained to identify features (e.g., marks 212, haptics 206) in the image, which may then be used to determine orientation of the IOL 200 programmatically or using another machine learning model.

The method 500 may include measuring, at step 508, corneal topography using the corneal topography device 406 and/or aberration using the aberrometer 410. The result of step 508 may be an estimate of the total refractive error of the eye 100, including the contribution of the IOL 200 to the refractive power of the eye 100. The total refractive error may include a measure of spherical error as well as a total astigmatism of the eye 100 (including the IOL 200) and axis of the astigmatism of the eye 100.

Using the image from step 504, the IOL identified in the image from step 506, the corneal topography and/or total aberration from step 508, a deficiency of the IOL 200 may be identified. As discussed below, the deficiency may be astigmatism (incorrect alignment of toric axis with axis of astigmatism and/or incorrect cylindrical correction), spherical error, or other refractive error of the eye 100 following placement of the IOL 200.

The method 500 may include characterizing, at step 510, corneal astigmatism, e.g., the contribution to astigmatism of the eye 100 resulting from astigmatism of the cornea 102. The corneal astigmatism may be obtained by evaluating the topology of the inner and outer surfaces 406a, 406b of the cornea 102 and modeling the refractive error of the cornea 102, specifically degree of corneal astigmatism and an axis of corneal astigmatism. Axial length between the cornea and the retina 108 along with the refractive power of the cornea 102 may provide the spherical error of the cornea 102.

The method 500 may include determining, at step 512, misalignment between the IOL axis 300 and the reference axis. For example, the orientation of the IOL axis 300 may be determined at step 506. A difference between the orientation of the IOL axis 300 and the axis of corneal astigmatism may therefore be used as the misalignment between the IOL axis 300 and the reference axis. Step 512 may further include evaluating whether the degree of astigmatism correction of the IOL 200 is correct. For example, the total degree of astigmatism of the combined eye 100 and IOL 200 may be compared to the resulting reduction of astigmatism if the IOL axis 300 were rotated to align with the axis of corneal astigmatism. If a difference between the total degree of astigmatism and the reduction is non-zero, or greater than some threshold, such as 0.25 diopters, then a different IOL 200 may be needed.

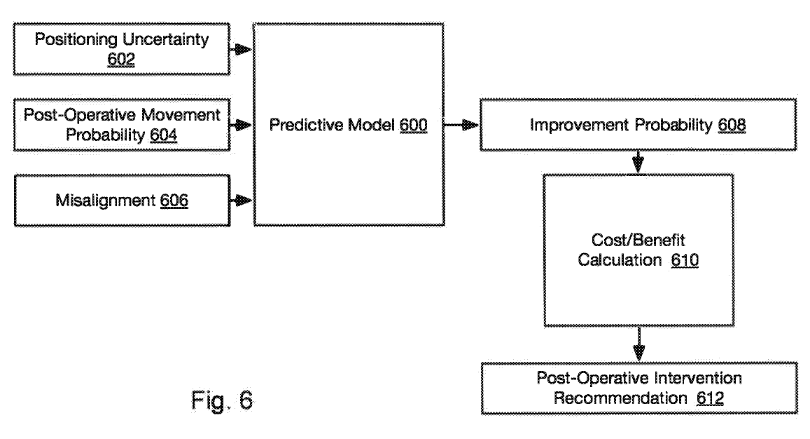

The method may include processing, at step 514, one or more items of data with a predictive model. The one or more items of data may include the misalignment and possibly the difference from step 512. Any spherical error of the combined eye 100 and IOL 200 may also be processed at step 514. The processing of step 514 may include determining a probability that the refractive error of the combined eye 100 and IOL 200 could be improved. The operation of the predictive model is described below with reference to FIG. 6.

A probability obtained from the predictive model may be processed at step 516 to obtain a post-operative intervention recommendation. For example, post-operative interventions may include rotation of the IOL 200 or placement of a new IOL 200. The processing of step 516 is likewise described below with reference to FIG. 6.

The post-operative intervention recommendation may specify whether an intervention is recommended, e.g., adjusting the orientation of the IOL 200, placement of a different IOL 200, not performing any intervention, or performing some other intervention. The post-operative intervention recommendation may be communicated to a user, such as by transmitting an email, text message, message to a client application, or output to a display device.

Referring to FIG. 6, a predictive model 600 may be used at step 514. The predictive model 600 may take as inputs a positioning uncertainty 602, a post-operative movement probability, and a misalignment 606, e.g., the misalignment from step 512. If a difference is found at step 512, e.g., insufficient degree of astigmatism correction, then this difference may also be used as an input. The positioning uncertainty 602 may be a statistical characterization of the ability of surgeons to position an IOL axis 300 relative to a reference axis 302. For example, the positioning uncertainty 602 may be a standard deviation of a distribution of positioning errors, 75^{th} (or other) percentile of positioning errors, or other metric of positioning error. Since positioning uncertainty may have a directional bias, a mean, median, or other statistical characterization of the center or other mode of the distribution of positioning errors may be included as well. The positioning uncertainty may include a positioning uncertainty along the optical axis of the eye 100 that would affect spherical error. The positioning uncertainty 602 may be a result of evaluating post-operative IOL orientation for many procedures by many surgeons or the same surgeon. The positioning errors may be final intra-operative positioning errors, e.g., measured during a procedure after a final adjustment of the IOL 200 but before the patient is transferred from under the ophthalmic microscope used during the procedure.

The post-operative movement probability 604 may be a statistical characterization of a shift between a final intra-operative positioning error of an IOL 200 and a post-operative positioning error, e.g., as measured after a healing period of a week, two weeks, or some other period. For example, the post-operative movement may be a standard deviation of a distribution of shifts, 75^{th} (or other) percentile of shifts, or other metric of shifts. Since post-operative shifts may have a directional bias, a mean, median, or other statistical characterization of the center or other mode of the distribution of shifts may be included as well. The post-operative movement probability 604 may also include a statistical characterization of axial shifts of the IOL 200 that result in spherical error. The post-operative movement probability 604 may include a statistical characterization of changes in corneal shape resulting in changes in astigmatism (axis of astigmatism and/or magnitude of cylindrical error) and/or spherical refractive error.

The predictive model 600 processes the inputs to derive an improvement probability 608, e.g., a likelihood that refractive error of the eye 100 and IOL 200 can be improved by an intervention, such as rotation of the IOL 200 or placement of a new IOL 200. The predictive model 600 may for example, use a Monte Carlo or other simulation technique along with the positioning uncertainty 602 and post-operative movement probability 604 to determine a distribution of likely outcomes, e.g., improvements. The improvement probability may be a statistical characterization of this distribution, such as the standard deviation, a 75^{th} or other percentile improvement value, mean, median, and/or other statistical characterization of the distribution.

The improvement probability 608 may be processed using a cost/benefit calculation 610 that evaluates the cost of an intervention with respect to the improvement probability 608 and possibly other factors, such as risk that may be a function of patient age, complications during an initial procedure, comorbidities, or other factors. The result of the cost/benefit calculation 610 may be a post-operative intervention recommendation 612, which may be a binary go/no go decision indicating whether the intervention is justified and may possibly include a report or other representation of inputs to the predictive model and/or the improvement probability.

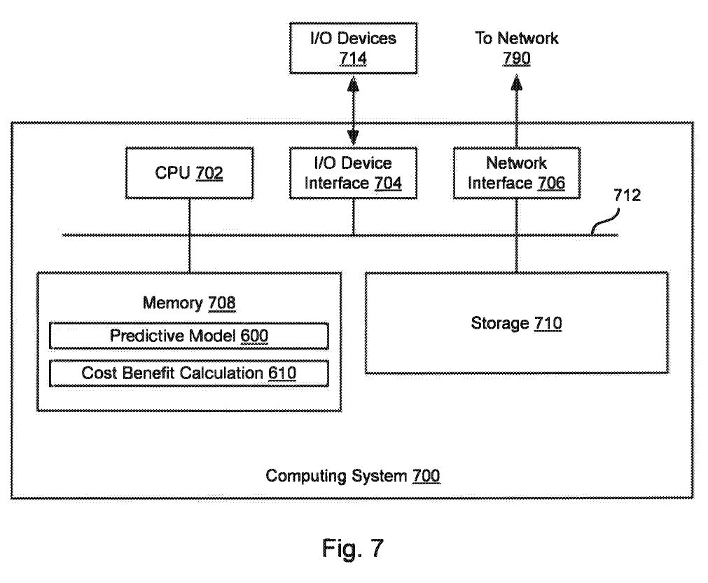

FIG. 7 illustrates an example computing system 700. The multi-modal imaging device 400 may have some or all of the attributes of the computing system 700.

As shown, computing system 700 includes a central processing unit (CPU) 702, one or more I/O device interfaces 704, which may allow for the connection of various I/O devices 714 (e.g., keyboards, displays, mouse devices, pen input, etc.) to computing system 700, network interface 706 through which computing system 700 is connected to network 790, a memory 708, storage 710, and an interconnect 712.

CPU 702 may retrieve and execute programming instructions stored in the memory 708. Similarly, CPU 702 may retrieve and store application data residing in the memory 708. The interconnect 712 transmits programming instructions and application data, among CPU 702, I/O device interface 704, network interface 706, memory 708, and storage 710. CPU 702 is included to be representative of a single CPU, multiple CPUs, a single CPU having multiple processing cores, and the like.

Memory 708 is representative of a volatile memory, such as a random access memory, and/or a nonvolatile memory, such as nonvolatile random access memory, phase change random access memory, or the like. As shown, memory 708 may store executable code implementing the predictive model 600, the cost/benefit calculation 610, and other functions described herein.

The computing system 700 may include storage 710, which may be non-volatile memory, such as a disk drive, solid state drive, or a collection of storage devices distributed across multiple storage systems.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for evaluating intra ocular lens (IOL) placement, the method comprising:
receiving, by a computing device, one or more images of an eye of a patient having an implanted IOL, the one or more images captured using one or more imaging devices having one or more imaging modalities;
determining, by the computing device, a deficiency of the IOL according to the one or more images;
processing, by the computing device, the deficiency using a predictive model to obtain an improvement probability indicating a likelihood that an intervention could decrease refractive error of the eye; and
outputting, by the computing device, an intervention recommendation according to the improvement probability.

2. The method of claim 1, wherein the one or more imaging devices include two or more imaging devices including at least two of:
a camera;
a corneal topography device;
an optical coherence tomography (OCT) device; or
an aberrometer.

3. The method of claim 2, wherein the two or more imaging devices are incorporated into a multi-modal imaging device.

4. The method of claim 1, further comprising capturing the one or more images using the one or more imaging devices while inducing red reflex from a retina of the eye of the patient.

5. The method of claim 1, wherein determining the deficiency of the IOL comprises determining misalignment of the IOL in the one or more images by detecting markings on the IOL in the one or more images.

6. The method of claim 1, wherein:
the one or more imaging devices include a visible light camera and a corneal topography device;
the one or more images include a first image captured using the visible light camera; and
the method further comprises:
determining an axis of corneal astigmatism according to an output of the corneal topography device;
determining an IOL axis of the IOL according to the first image; and
determining the deficiency by determining a misalignment as a difference between the IOL axis and the axis of corneal astigmatism.

7. The method of claim 6, wherein processing the deficiency using the predictive model comprises processing the misalignment along with a positioning uncertainty and a post-operative movement probability using the predictive model.

8. The method of claim 6, wherein processing the deficiency using the predictive model comprises processing the misalignment along with a positioning uncertainty and a post-operative movement probability according to a Monte Carlo simulation.

9. The method of claim 1, wherein outputting, by the computing device, the intervention recommendation according to the improvement probability comprises performing a cost benefit analysis with respect to the improvement probability.

10. The method of claim 1, wherein the intervention recommendation is a recommendation to rotate the IOL.

11. The method of claim 1, wherein the deficiency is at least one of a spherical refractive error or a cylindrical error and the intervention recommendation is a recommendation to replace the IOL.

12. The method of claim 1, wherein the IOL is a toric IOL.

13. A system for evaluating intra ocular lens (IOL) placement, the system comprising:
one or more imaging devices; and
a computing device configured to:
receive one or more images of an eye of a patient having an implanted IOL, the one or more images captured according to one or more imaging modalities;
determine misalignment of the IOL according to the one or more images;
process the misalignment using a predictive model to obtain an improvement probability indicating a likelihood that an intervention could decrease refractive error of the eye; and
output an intervention recommendation according to the improvement probability.

14. The system of claim 13, wherein the one or more imaging devices include two or more of:
a camera;
a corneal topography device;
an optical coherence tomography (OCT) device; or
an aberrometer.

15. The system of claim 14, wherein the two or more imaging devices are incorporated into a multi-modal imaging device.

16. The system of claim 13, wherein the one or more imaging devices are configured to capture the one or more images using the one or more imaging devices while inducing red reflex from a retina of the eye of the patient.

17. The system of claim 13, wherein:
the one or more imaging devices include a visible light camera and a corneal topography device;
the one or more images include a first image captured using the visible light camera; and
the computing device is further configured to:
determine an axis of corneal astigmatism according to an output of the corneal topography device;
determine an IOL axis of the IOL according to the first image; and
determine the misalignment as a difference between the IOL axis and the axis of corneal astigmatism.

18. The system of claim 13, the computing device is further configured to process the misalignment using the predictive model by processing the misalignment along with a positioning uncertainty and a post-operative movement probability using the predictive model.

19. The system of claim 13, the computing device is further configured to process the misalignment using the predictive model by processing the misalignment along with a positioning uncertainty and a post-operative movement probability according to a Monte Carlo simulation.

20. The system of claim 13, the computing device is further configured to perform a cost/benefit analysis with respect to the improvement probability.
